(19) United States
(12) Patent Application Publication
(10) Pub. No.: US 2019/0014243 A1
(43) Pub. Date: Jan. 10, 2019

(54) Telecentric Zoom Lens System For Optical Capture Of A Generated Event

(71) Applicant: National Security Technologies, LLC, Las Vegas, NV (US)

(72) Inventors: Robert M. Malone, Los Alamos, NM (US); Morris I. Kaufman, Los Alamos, NM (US)

(73) Assignee: National Security Technologies, LLC, Las Vegas, NV (US)

(21) Appl. No.: 14/209,986

(22) Filed: Mar. 13, 2014

Related U.S. Application Data
(60) Provisional application No. 61/780,669, filed on Mar. 13, 2013.

(51) Int. Cl. H04N 5/225; G02B 9/04; H04N 5/235; A61B 6/00; G02B 7/04; G02B 13/22

(52) U.S. Cl. CPC H04N 5/2254; G02B 9/04; G02B 13/22; A61B 6/52; G02B 7/04; H04N 5/2353

(57) ABSTRACT

A method and apparatus for detecting and recording an event is disclosed which includes a scintillator located to receive energy from the event. The energy may be x-ray energy from a radiographic source. A mirror is optionally angularly located adjacent the scintillator to redirect or reflect the light into two or more lenses. The two or more lenses have zoom capability to adjust the size of the image and the two or more lenses are arranged as telecentric lenses. A camera receives the light presented from the two or more lenses on an image plane to establish a recorded image while a shutter is presented to selectively control presentation of the light to the camera. The zoom lens allows use of different size scintillators and various CCD cameras to benefit from multiple zoom positions to best capture the image generated by the scintillator.

### PRIORITY CLAIM

This application claims priority to and the benefit of U.S. Provisional Patent Application No. 61/780,669 filed on Mar. 13, 2013.

### STATEMENT REGARDING FEDERAL RIGHTS

This invention was made with government support under Contract No. DE-AC52-06NA25946 and was awarded by the U.S. Department of Energy, National Nuclear

Security Administration. The government has certain rights in the invention.

### FIELD OF THE INVENTION

The invention relates to a lens system and in particular to a telecentric zoom lens system at finite conjugate distances for use with a scintillator light source.

### RELATED ART

Optic systems are known in the art to convey optic signals from a source to a camera or other recording or viewing screen. The source of the optic signals can be any source, but one example field of use is for experiments or generated phenomena that are generated by a signal source. One example of a signal source is a chamber or other containment vessel in which an event may occur. The event may be a high-energy pulsed radiographic x-ray source. In this source, a rod-pinch x-ray diode, or other source, produces a point source having a diameter, such as for example, 1 mm diameter. The x-rays are directed at a target object. In such a source, the target object is placed at a distance, such as 1.5 meter, from the x-ray source, and a large scintillator is placed at a further distance, such as at 2.4 meters. The scintillator type may be a LYSO scintillator LYSO (Lu_{1.8}Y_{0.2}SiO₅(Ce)) or any other type material or configuration.

It is contemplated that different-sized or type objects may be imploded within the containment vessel and the event recorded. To record the events, a lens system directs optic signals from the event to a camera or other recording or observation device. Prior art lenses and capture devices suffer from several drawbacks. One such drawback was that the existing lenses do not meet a telecentric requirement. The telecentric condition occurs when light rays emitted from any of the field points of the emitter are all parallel to each other. Prior art lenses collect light rays at various angles from different field points of an emitter. Prior art lenses collect good images from a surface emitter but do not work well with a volume emitter. Another drawback is that normal lenses have no zoom capability. Thus, when different scintillators and/or cameras are used with normal lenses, optimal resolution is not collected from the scintillator. This is a significant drawback and results in the camera being either under filled or over filled.

Another drawback to prior art lens system was that the resolution was less than desired and light collection and transmission left the resulting image light starved. Prior art lenses also resulted in undesirable signal to noise statistics of the recorded image. The innovation described below overcomes these drawbacks in the prior art.

### SUMMARY

To overcome the drawbacks of the prior art, a system is disclosed for recording an event comprising a scintillator located to receive energy from the event. A lens group that includes two or more lenses, receives the light from the scintillator and directs the light to a camera. The camera is configured to capture and record the light directed through the lens group from the scintillator to thereby record an image of one or more aspects of the event. The lens group includes two or more lenses and at least one lens is movable, relative to another of the two or more lenses, to enable a change in magnification of the image represented by the camera.

In one embodiment the event comprises generation of x-ray energy. In one configuration a mirror presented at an angle to direct the light from the scintillator to the lens group. The mirror may be a pellicle mirror. It is contemplated that the lens group may comprise a first lens group configured to receive the light from the pellicle such that the first lens group comprising one or more lenses, and a first doublet lens configured to receive the light from the first lens group and a second doublet lens configured to receive the light from the first lens group, such that the first doublet lens can move relative to the second doublet lens.

The system may further comprise a shutter configured to selectively establish an aperture to electively pass and block light traveling from the lens group to the camera. The shutter may comprise a mechanical shutter. The scintillator may comprises a LYSO scintillator or a Csl(Tl) scintillator. Csl is sesium iodide (thallium doped). In addition, the lens group may be telecentric.

Also disclosed is a method for recording an event comprising the steps of receiving energy from an event at a scintillator, the scintillator converting the energy to a light at a visible wavelength. Then, receiving the light along an light path axis at a first lens group such that the first lens group comprises one or more fixed lenses and then presenting the light from the first lens group to first zoom lens element and a second zoom lens element. At least one of the first zoom lens element or a second zoom lens element is movable in the direction the light path axis. The light is then received at an image plane of a camera and the light forms an image on the image plane such that the movement of the first zoom lens element or a second zoom lens element adjusts the size of the image on the image plane. The camera can then record the image at a selected magnification.

In one embodiment, the energy comprises x-ray energy and the scintillator converts the x-ray energy to a visible wavelength. The scintillator may comprise or consist of a LYSO scintillator or a Csl(Tl) scintillator. This method of operation may further comprise reflecting the light from the scintillator into the first lens group with a pellicle mirror. It is optional to actuate a shutter to selectively present the light to the camera.

Also disclosed is an imaging system for use with a pulsed radiographic x-ray source that includes a first lens group configured to receive light in the form of x-ray energy. A second lens group is also present and it has zoom capability, the second lens group having at least one lens that is moveable in relation to one or more other lenses in the second lens group. A shutter is configured to selectively pass the light to a camera which is configured to capture the light to form an image such that the image represents x-rays from the radiographic x-ray source.

In one embodiment, the light is generated by a scintillator which generates light in response to contact by x-ray radiation. The camera may comprise a 62 mm CCD camera and is movable along an axis, the axis parallel to the direction of the light from the second lens group. It is contemplated that the first lens group and the second lens group may establish a telecentric lens system.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

The components in the figures are not necessarily to scale, emphasis instead being placed upon illustrating the principles of the invention. In the figures, like reference numerals designate corresponding parts throughout the different views.

FIG. 1 is a perspective view of an exemplary event chamber with two lens assemblies and cameras.

FIG. 2 is a perspective view of the scintillator, pellicle, lens assembly and camera.

FIG. 3 illustrates a perspective view of an exemplary rail system for supporting and securing the elements shown in FIG. 2.

FIG. 4 illustrates various fields of view at the scintillator for different zoom options.

FIG. 5 illustrates a side plan view of the lens assembly.

FIG. 6 illustrates a side plan view of the lens assembly with an emphasis on components that move during magnification changes.

FIG. 7 shows the maximum extent of movement for the adjustable components of the lens system.

FIG. 8 illustrates exemplary lens assembly tolerance values.

FIG. 9 illustrates a side plan view of the camera in relational position to a lens group for zoom position 5.

FIG. 10 shows a perspective view pellicle positioning in relation to the scintillator and the first lens element.

FIG. 11 illustrates an alternative embodiment of a lens system using a Csl(Tl) scintillator for operation at green wavelengths.

FIGS. 12A-12D illustrate the lens system at different zoom or magnification steps to accommodate different fields of view.

### DETAILED DESCRIPTION

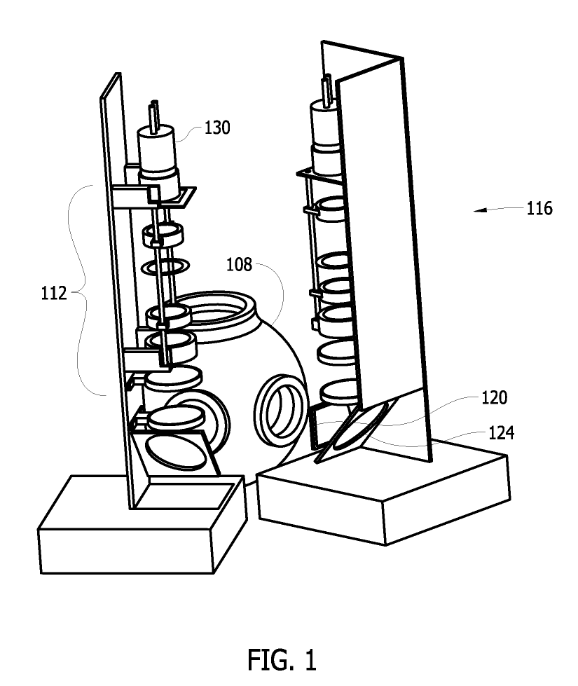

FIG. 1 is a perspective view of an exemplary event chamber with two lens assemblies and camera. This is but one possible field of use for the lens system shown and described herein. In this embodiment, a chamber 108 is provided to contain an event. The event may be an explosion, implosion, pulsed x-ray event, calibration, or any other type event or occurrence in which observation or recordation of the optic signal is desired. X-ray signals representing the event exits the chamber 108 and encounters a scintillator 120 or any other plate or surface. A scintillator is a material that exhibits luminescence when excited by ionizing radiation. Luminescent materials, when struck by an incoming particle, absorb its energy and scintillate, (i.e., re-emit the absorbed energy in the form of light). Sometimes, the excited state is metastable, so the relaxation back down from the excited state to lower states is delayed (necessitating anywhere from a few microseconds to hours depending on the material).

A large pellicle 124 deflects the scintillator light out of the x-ray path into a zoom lens array 112, 116 coupled to a CCD camera 130. In one other embodiment, other mirrors may be utilized other than a pellicle 124, or the lens system 112 may align directly with the chamber. In one embodiment the camera 130 comprises a CCD type camera but in other embodiments any type camera may be used. In this embodiment, an eleven-element zoom lens system 112 is selected, but in other embodiment, other number of lenses may be used.

In this embodiment, the zoom lens 112 and camera 130 are as close as possible to the scintillator to maximize light collection, but in other embodiments the distance may vary. As a benefit of this embodiment, the lens system 112 is a telecentric lens design configured to minimize image blur from a volume source. To maximize the resolution of objects of different sizes, the scintillator 120 and zoom lens 112 are translated along the x-ray axis to change the zoom lens magnification.

Additional support structure is shown to support and secure the scintillator 120, pellicle 124, lens assembly 112, and the camera 130. Any support structure may be used as is discussed herein and as such, the support structure is not described in detail.

In this embodiment, the lenses weight 60 lb and are located in a 340 lb mechanical structure which is orientated vertically as shown in FIG. 1. In one example environment of use, the lens assembly, scintillator, and camera is used with Cygnus. Cygnus is a high-energy (2.25 MeV) radiographic x-ray source producing 50 ns FWHM pulses. A rod-pinch x-ray source produces a 1 mm diameter spot size while x-ray collimators define the beam axis. At the scintillator position, the x-rays appear to come from a small point source. The limiting resolution of the imaging system may be dominated by the finite size of this rod-pinch source.

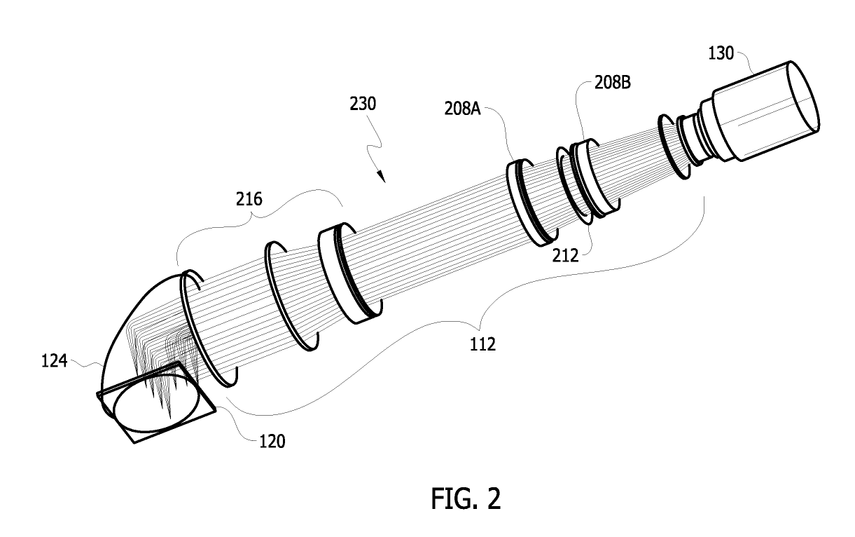

FIG. 2 is a perspective view of the scintillator, pellicle, lens assembly and camera. This figure is helpful in showing one example relational position of the scintillator 120, pellicle 124, lens assembly 112 and camera 130. The x-rays or other energy exiting the chamber (shown in FIG. 1) encounters the scintillator 120 which fluoresces or otherwise emits an optic signal which is reflected by the pellicle 124 to the lens assembly 112. The lens assembly 112 is discussed in detail below. The lens assembly, which may be configured as a telecentric zoom lens system, directs the light to the camera 130 or other recording or observation device.

In this configuration the zoom magnification is changed when different-sized recording cameras are used, such as for example a 50 or 62 mm square format camera. In other embodiments, other camera sizes may be adopted for use.

In this embodiment, the scintillator 120, which may be a LYSO type scintillator, measures 200×200 mm and is 5 mm thick. In other embodiments other sizes and thicknesses of scintillator 120 may be used, or another medium other than a scintillator may be placed between the event and the pellicle or lenses. The pellicle 124 should be considered an optional element. In this embodiment, the scintillator 120 produces blue light peaking at 435 nm, so lens materials tailored to this wavelength is preferred. In other embodiments, other wavelengths may be used and different corresponding lens material utilized. For example, the zoom lens can also use a Csl(Tl) scintillator that produces green light centered at 540 nm.

This configuration has the benefit of at least one new lens element and at least one lens may be configured to move. All lenses in both wavelength bands may have antireflective (AR) coating.

In this configuration, there are two sets of doublet lenses 208A, 208B, and a stop lens 212. These lens elements 208A, 212, 208B and the camera 130 are configured to move during zoom operations. One doublet (208A or 208B) may be configured with XY compensation. A set of lenses 216 resides between the doublet lens 208A and the pellicle 124. The lens group 216 may use fused silica for radiation damage control.

Also shown in FIG. 2 and other figures are lines 230 representing emission from discrete field points of the scintillator. These lines 230 are traced to the camera 130. Stray light analysis (not shown here) would reveal that the unwanted light from the scintillator is blocked. In one embodiment the zoom lens system collects 0.1 NA light from the scintillator, which in turn emits light with much larger NA.

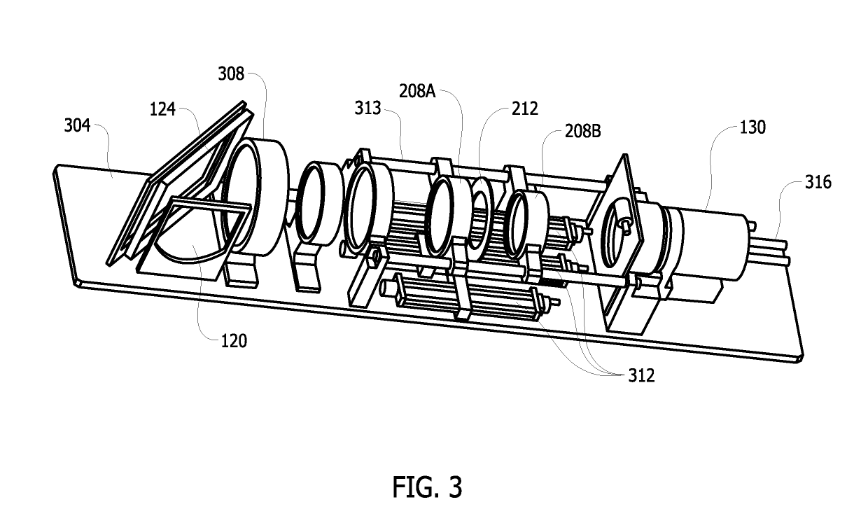

FIG. 3 illustrates a perspective view of an exemplary support system for supporting and securing the elements shown in FIG. 2. Some of the elements of the rail system are movable, such as elements 208A, 212, 208B and the camera 130. The rail system includes a base 304 to which one or more lens supports 308 may fixedly and removably connect. In the event some lenses are movable, the support system may include rails 312 along which one or more lenses 208A, 212, 208B may move. Camera rails 316 are also provided to allow for movement of the camera 130. Magnetic encoders 313 provide absolute position information for each moveable component.

In one embodiment the rail system for moving the optical components makes use of a magnetic encoder 313 that doesn't require power to maintain its value. In other embodiments other automatic or manual rail systems may be used. In one example environment of use of the zoom lens system described herein is configured to allow for a 20° temperature difference between winter and summer operations. This may affect system design and tolerances.

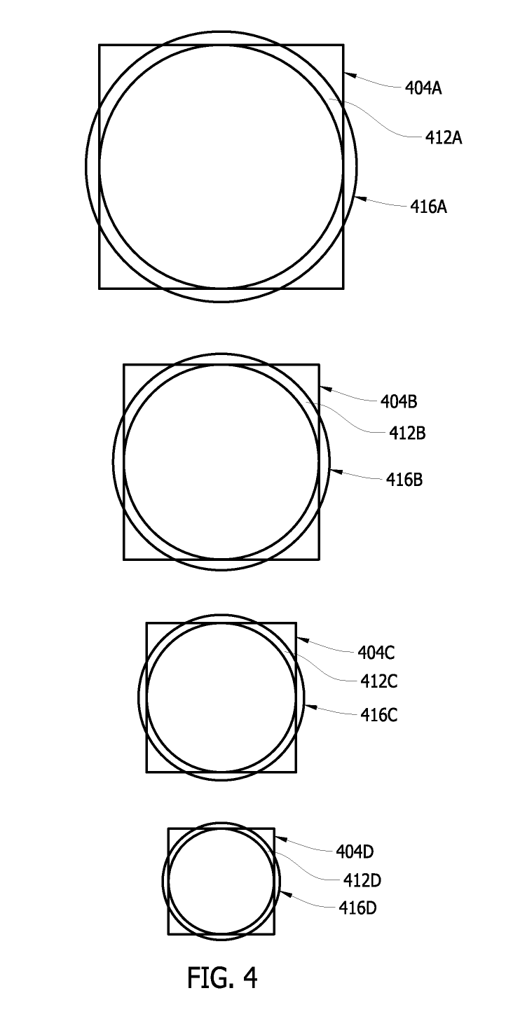

FIG. 4 illustrates various layouts for zoom options. As a benefit of the zoom lens disclosed herein, this zoom lens is designed to image different-sized scintillators. The outer dimension of the scintillators 404 are shown in FIG. 4 over the optical zoom coverage 412 of the camera. However, there is only one scintillator in use, and the field of view is adjusted depending on the object size to be x-rayed and possibly the size of the scintillator. It is possible to place calibration markers (fiducials, step wedges, etc.) outside the field of view. In reference to the discussion below, odd zoom positions make use of a 62 mm diameter CCD camera while even zoom positions use the 50 mm diameter camera.

In this configuration, zoom positions 1 and 2 show a square scintillator material 404A having measurements of 200 mm by 200 mm. A 10 mm thick calibration zone 412A is provided while a 220 mm diameter object field of view 416A is available to be imaged. For zoom positions 3 and 4, shown is a square scintillator material 404B having measurements of 160 mm by 160 mm. A 10 mm thick calibration zone 412B is provided while a 180 mm diameter object field of view 416B is available to be imaged. For zoom positions 5 and 6, shown is a square scintillator material 404C having measurements of 120 mm by 120 mm. A 10 mm thick calibration zone 412C is provided while a 140 mm diameter object field of view 416C is available to be imaged. For zoom positions 7 and 8, a square scintillator material 404D having measurements of 85 mm by 85 mm is shown. A 10 mm thick calibration zone 412D is provided while a 105 mm diameter object field of view 416D is available to be imaged.

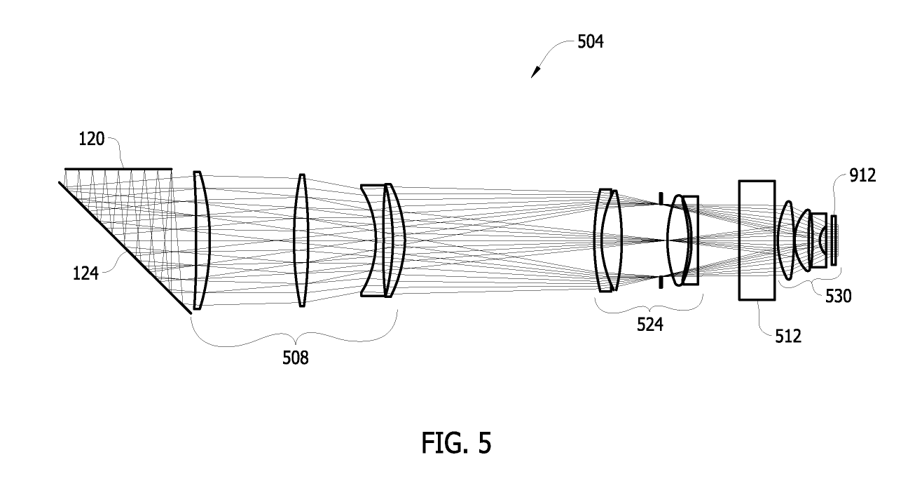

FIG. 5 illustrates a side plan view of the lens assembly. This is but one possible arrangement of lens and as such, other configurations are possible. Before moving to the details of FIG. 5, a general discussion of the lens system is provided. Although the lens system described herein is defined as a zoom lens, it differs from a traditional zoom lens in a number of ways. In a typical zoom lens the back focal distance is fixed, the detector size is fixed, and the front focal distance is variable (object distance). For this new lens system, the back focal distance is allowed to vary as a compensator for camera focus and several detectors have been considered so the image size may vary. While the front focal distance is fixed (distance to scintillator) the size of the scintillator may vary considerably. Because of x-ray collimation on the scintillator, the new lens can be telecentric, meaning that the center ray of each ray bundle emanating from a point on the object must be parallel to the optical axis. The peculiar combination of requirements made this a challenging optical design problem.

Another novel aspect of this design is that it is a telecentric zoom lens. This means that the light is collected along the trajectory that the x-ray makes upon passing through the scintillator. Each x-ray generates light continuously along its track through the scintillator. A telecentric lens allows for light collection from a volume emitter without loss of resolution. A volume emitter is a scintillator that has thickness greater than the depth of focus of its light collecting lens. Normal lenses collect light at different angles from an object. With a prior art lens there will be image blur if the object light source is too thick. This occurs because prior art lenses work poorly when the scintillator thickness increases. Because pulsed x-ray imaging of dense targets may be starve for light, thicker scintillators are desirable. The zoom feature of this innovation means that different-sized scintillators and/or different-sized CCD cameras can be used, whereas this was not possible in the prior art.

As part of the design process and improvements, several considerations were made regarding how many lenses would be required to achieve the design constraints. For example, it was considered whether aspheric lenses be used to reduce the number of elements. Another design challenge was determining the number of elements required to move during the zoom operation when the magnifications were changed.

Turning now to FIG. 5, shown is a lens layout 504 for zoom position #1 as shown in FIG. 4. In this embodiment, the first three elements 508 use an Ohara version of fused silica to gather the light from the scintillator. A mechanical or any other type of shutter 512 blocks the event, such as high explosive light after x-ray event time or other energy thereby preventing unwanted background light during the camera read time. The elliptical pellicle 124 is decentered by 16.5 mm to center the footprint of light reflected off its coating, which in this embodiment is aluminum. Movable lens elements 524 are arranged after the first lens group 508 but before the shutter 512. Behind the shutter 512 is a three element lens group 530. An image capture device 130 (not shown), such as a camera is configured to receive the optic signal. The vacuum window 912 is part of the camera system 130. Various dimension are shown in FIG. 5, but are provided for purposes of understanding and enablement. The claims that follow are not limited to these exemplary dimensions.

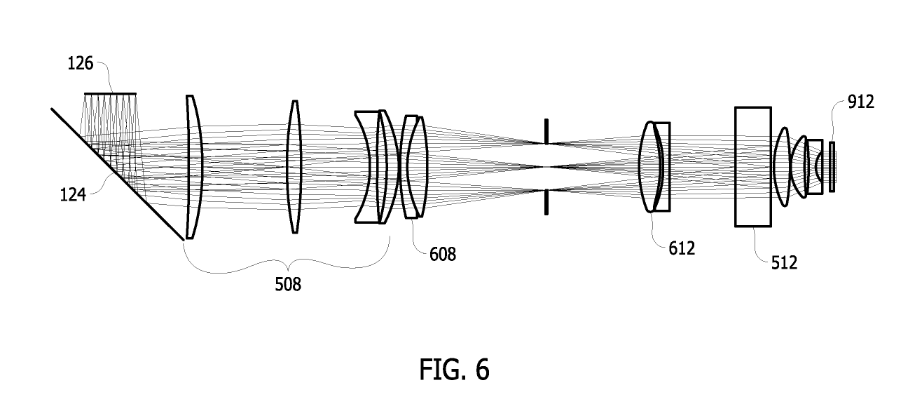

FIG. 6 illustrates a side plan view of the lens assembly with an emphasis on components that move during magnification changes. As compared to FIG. 5, similar elements are identified with identical reference numbers. This layout is for zoom position #7 using an 85 mm square scintillator, 105 mm diameter field of view, recording onto a 62 mm diameter camera. The collected light makes a much smaller footprint on the pellicle. For each zoom position, the diameter of the stop changes, using a series of plates with different diameter holes cut out of them.

In this figure, movable doublets are lens elements 608 and 612. The mechanical shutter 512 does not move. The zoom lens operation occurs such that the movement of the magnifier lens group 608 changes image magnification for different scintillators, while movement of the adjuster lens group 612 compensates for aberration induced by the magnification change. In this configuration the stop size and position are also allowed to vary but in other embodiment these elements could be fixed. The camera is allowed to refocus through movement on rails or other movement or focusing means.

In one embodiment, the two moving doublets 608, 612 may have their elements cemented together to improve resolution performance. However, cementing large optical elements together may result in differing thermal expansion of these dissimilar large glasses which may undo the cement resulting in both exposed glue and uncoated surfaces causing deteriorated optical performance.

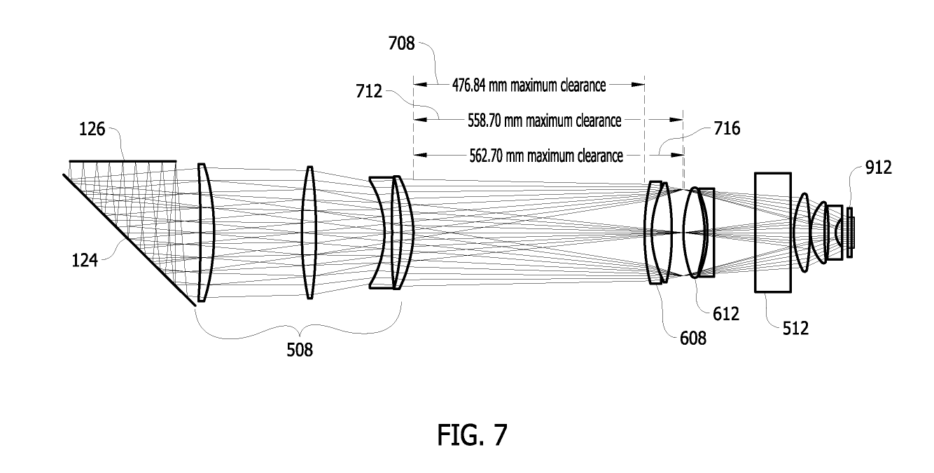

FIG. 7 shows the maximum extent of movement for the adjustable components of the lens system. As compared to FIGS. 5 and 6, similar elements are identified with identical reference numbers. Maximum movement occurs in zoom position #2, which uses the 200 mm square scintillator, 220 mm diameter field of view, and the 50 mm diameter camera 130. As can be seen in comparison to FIG. 6, the double lens 608 is physically close to doublet lens 612. Shown in FIG. 7 are the maximum distances 708, 712, 716. In this example embodiment, the maximum distance 708 is about 478.84 mm while the minimum distance (not shown) is about 2 mm. In this example embodiment, the maximum distance 712 is about 558.70 mm while the minimum distance (not shown) is about 309.07 mm. In this example embodiment the maximum distance 716 is about 562.70 mm while the minimum distance (not shown) is about 502.11 mm. The minimum movement occurs in zoom position #7 which uses an 85 mm square scintillator, 105 mm diameter field of view, and the 62 mm diameter camera.

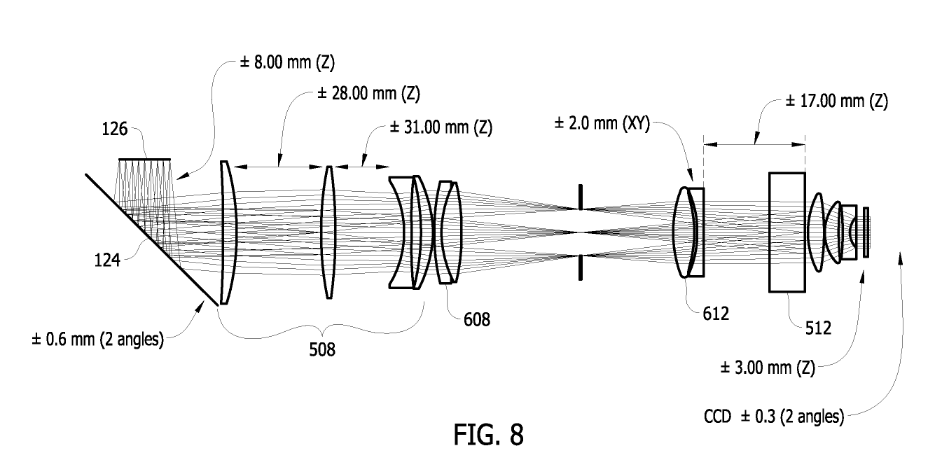

FIG. 8 illustrates exemplary lens assembly tolerance values. As compared to FIGS. 5, 6, and 7, similar elements are identified with identical reference numbers. This is but one arrangement and associated tolerance values and as such, in other embodiments other tolerance values may be established. The exemplary tolerances are shown in FIG. 8 and as such not repeated herein. Analysis of the tolerances of the zoom lens was conducted in order to achieve best resolution. In one configuration, the second moveable doublet is capable of an XY adjustment of only ±2.0 mm. Without this small adjustment, which compensates for lens manufacturing errors, some of the zoomed positions have close to zero Modulation Transfer Function (MTF) at 16 lp/mm. The large tolerance values for some of the z-axis motions of the optical elements are worst case values. During construction, polishing may occur to improve optical performance, but this may make the lens thinner. Having a thick tolerance value allows for many re-polishing operations before the lens becomes too thin and has to be discarded. The thickness tolerance of each lens is ±2.0 mm. Most lenses have the thickest possible value. The spacing tolerances are actual compensators used to optimize resolution. Their exact tolerance values will change during assembly and optimization.

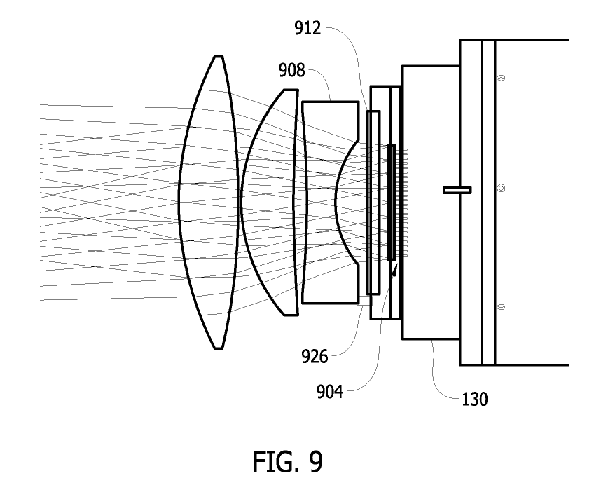

FIG. 9 illustrates a side plan view of the camera in relational position to a lens group for zoom position 5. In this figure, the lens 908 closest to the camera is configured to contact a limit switch 904 (or the camera contact the limit switch) which are carefully placed to prevent the CCD 904 of the camera assembly 130 from contacting the zoom lens 908 which could result in damage to the CCD 904 of the camera 130 or the lens 908. A window 912 may also be provided to replace or supplement the camera's glass cover to provide a vacuum seal. In one embodiment, the window 912 is 7 mm thick. The window 912 moves with the camera 103 assembly.

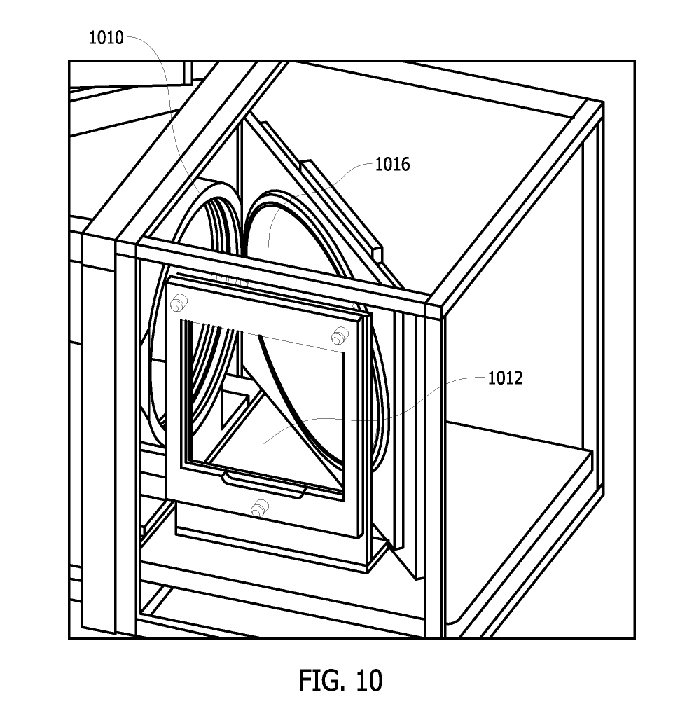

FIG. 10 shows a perspective view of the pellicle positioning in relation to the scintillator and the first lens element. The scintillator 1012 is shown as generally perpendicular to the lens 1010 while the pellicle 1016 is at an angle to reflect the optic signal from the scintillator 1012 to the lens 1010. The elliptical pellicle may be mounted into a tip/tilt frame. The scintillator may also be configured with tip/tilt adjustments. In this embodiment, the first lens 1010 does not tilt, but in other embodiments the first lens may tilt or tip. In this configuration, the pellicle 1016 is oversized to minimize edge effects of the bounding of the aluminized Mylar to its frame. The pellicle mount may be pinned to the base plate for removal and repositioning. Because there is no metal behind the pellicle, x-rays have no opportunity to scatter off it. The pellicle mount is specifically designed to minimize any x-ray scatter that could add background fluorescence light to the image if the scattered x-rays are deflected into the lens elements.

In one embodiment, an LED and divergent lens is positioned under the far corner of the pellicle mount and is pointing to the center of the scintillator 1012. The LED may be a blue LED or another color. This will be used during dry runs of the recording equipment without the Cygnus x-ray source. An array of resolution bars and step wedge features may line the outside of the field of view, as described in FIG. 4.

In another embodiment, four LEDs are attached to the frame of lens 1010. Their light reflects off the pellicle and onto the scintillator and its calibration features. This provides a light source for dry runs of the imaging system without use of the Cygnus pulsed x-ray source.

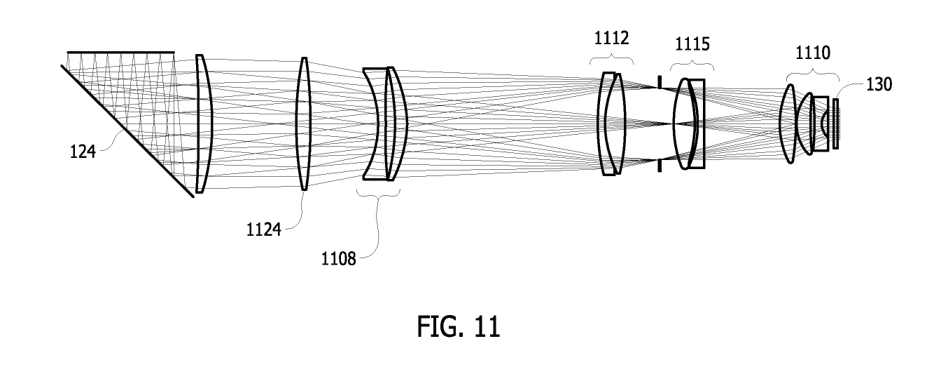

FIG. 11 illustrates an alternative embodiment of a lens system using a Csl(Tl) scintillator for operation at green wavelengths. The Csl(Tl) scintillator emits green wavelengths of light. The prior art optical systems have poor resolution when changing the spectrum from blue to green. To determine which lens element is most sensitive to the change in wavelength, systematic glass material changes to one lens at a time were examined to determine which lens element was the most sensitive to correcting resolution. It was found that only one lens element needed its glass material changed.

It was determined that the LYSO scintillator has a peak emission at 440 nm while the Csl(Tl) has its peak emission at 540 nm. In order to accommodate future uses of this zoom lens, one or more optical elements may be AR (anti-reflectivity) coated for 390-700 nm wavelengths, with the peak transmission at 500 nm. FIG. 11 shows zoom lens system modifications that will allow operation at green wavelengths. In the embodiment shown, one glass element was changed and positions of all lenses have changed. As shown the pellicle 124 and the camera 130. Between the pellicle 124 and the camera130 are the lenses. The fixed positions of lens elements 1124, 1108, 1110 shift position to accommodate compatibility with a Csl(Tl) scintillator. This requires different mounting holes to the supporting base plate 304. The elements 1112 and 1115 are the movable doublets, and one of the glass lenses inside element 1115 is a new lens tailored for use with the Csl(Tl) scintillator.

The lens system has several benefits over the prior art. When the x-rays are emitted from a point source of the Cygnus machine the light rays are nearly collimated passing through the thickness of the LYSO scintillator. Light is continuously produced along each x-ray track as it passes through the scintillator. However, the prior art lens system collects light at an angle to the x-ray axis. As the scintillator thickness increases, there is increased blurring of the collected image. To overcome this drawback in the prior art, use of telecentric zoom lens collects light along the direction of the x-ray axis. The prior art lens has a fixed working distance from the scintillator to its first optical element. Thus, there is a fixed magnification for the prior art imaging system, which does not provide the benefits of a zoom lens system. For example, prior art lens systems collected light from a 150 mm diameter scintillator and imaged it onto a 50 mm diameter CCD camera. But, if it is desired to use a higher-resolution CCD camera that has a 62 mm diameter, you will collect light into only 50 mm of the 62 mm diameter area available. Thus the prior art lens system was not using the available resolution of the camera. Through use of a zoom lens as proposed herein, the magnification can be changed, allowing many different camera sizes to be used.

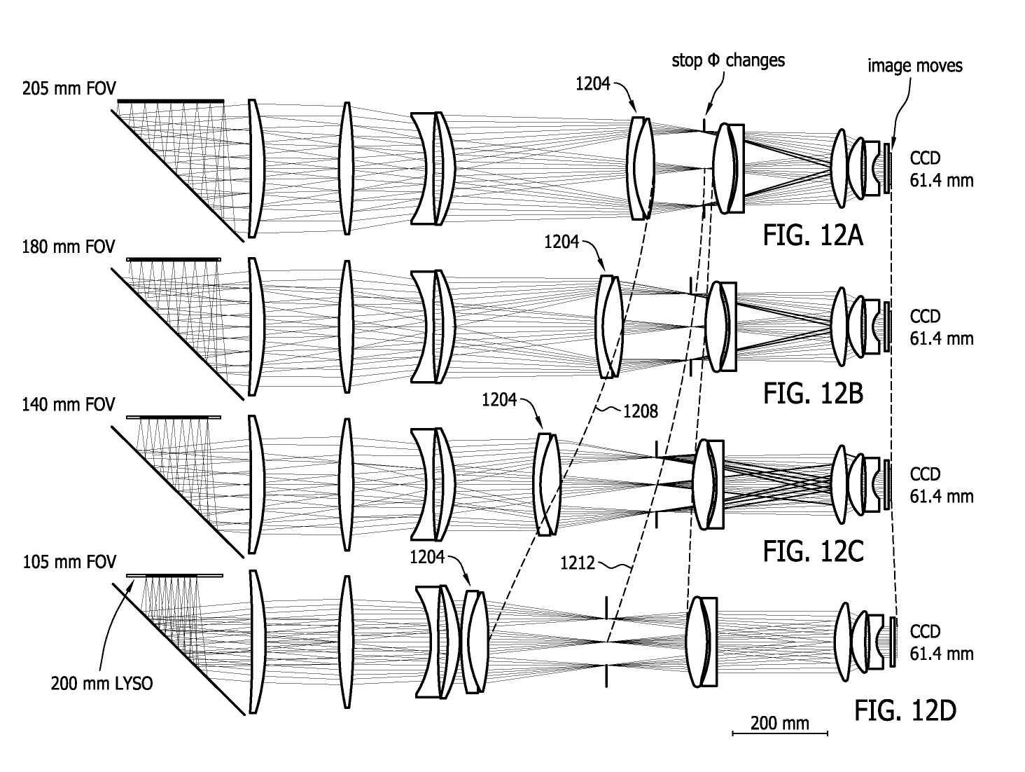

FIGS. 12A-12D illustrates the lens system at different zoom or magnification steps to accommodate different field of views. FIGS. 12A-12D are discussed together due to the overlapping nature of these figures. As shown in FIG. 12A, magnifier lens group 1204 is at a first position based on a 205 mm field of view (FOV). In FIG. 12B, the magnifier lens group 1204 is move to a second position based on a 180 mm FOV to accommodate a different size of image on the scintillator. As can be seen in FIG. 12B, the back edge lens position 1208 moves with the movement of the magnifier lens group 1204. As shown, the stop position 1212 also moves with each change in the magnifier lens group 1204 position.

Turning to FIG. 12C, the magnifier lens group 1204 is moved to a third position to accommodate a 140 mm FOV. FIG. 12D illustrates the lens system with the magnifier lens group 1204 located at a fourth position to accommodate a 105 mm FOV. This position is the smallest field of view for this configuration due to the location limitations established by other lenses in the lens system. In other embodiments, other configuration may allow movement of the magnifier lens group 1204 to different locations to accommodate other fields of view. In addition, it is contemplated that the magnifier lens group 1204 may be located at any location between the first position shown in FIG. 12A and the fourth position shown in FIG. 12D. In yet other embodiments, the entire lens system may be modified yet still maintain a telecentric lens system with zoom or magnification capability at finite conjugate distances.

In one embodiment, correct focus adjustments for the telecentric zoom lens system were optimized. To set the focus correctly for a volume emitting scintillator, a special calibration plate could be inserted into the scintillator holder. This holder is designed to swap scintillators and calibration plates and still maintain correct tip/tilt alignments with the x-ray axis. These calibration plates contained an array of small Air Force resolution patterns which are used to measure resolution at different locations across the scintillator area. Different thicknesses of Sapphire windows were laid on top of some of these Air Force resolution patterns. The different thickness Sapphire windows simulated different planes of focus within the scintillator. Sapphire has almost the same index of refraction as the LYSO or Csl(Tl) scintillators. Using this technique, the CCD camera could be precisely focused at the middle of the scintillator volume emitter. After best focus was optimized using the calibration plate, the plate was exchanged for the proper scintillator.

To prove the benefit of the telecentric light collection lens versus prior art lenses, a dense target with a matrix of very small holes was inserted into chamber 108. Cygnus x-rays passed through this target to produce a matrix of very small light emitting spots at the volume emitting scintillator. Images of these dots recorded from the telecentric zoom lens were compared to images recorded on a prior art lens. For the prior art lens, the ellipticity of the dots increased as the position of the dot changed from the scintillator center to the scintillator edge. The telecentric lens system showed no change in ellipticity for any of the dots from the center position to the edge of the scintillator. If the scintillator were very thin, both the telecentric lens system and the prior art lens would both show uniform circular dots across the field of view. However, as the thickness of the scintillator increases, the prior art lens would show increasing ellipticity of the dot images at the edge of the scintillator.

The zoom lens proposed herein is much larger and capable than the prior art lens. The prior art lens shared the same vacuum as the CCD camera. This shared vacuum seal between these two components did not allow any focus adjustment in the prior art system. In the embodiment disclosed herein, the zoom lens is completely detached from the CCD camera, allowing for focus adjustments. In other embodiment, it could share the same vacuum, but that would prevent focusing.

While various embodiments of the invention have been described, it will be apparent to those of ordinary skill in the art that many more embodiments and implementations are possible that are within the scope of this invention. In addition, the various features, elements, and embodiments described herein may be claimed or combined in any combination or arrangement.

What is claimed is:

1. A system for recording an event comprising:
a scintillator located to receive energy from the event;
a mirror located adjacent the scintillator and presented at an angle to reflect light from the scintillator;
a lens group comprising two or more lenses;
a camera configured to capture and record the light directed through the lens group from the scintillator to thereby record an image of one or more aspects of the event, such that the lens group includes two or more lenses and at least one lens is movable, relative to another of the two or more lenses, to enable a change in magnification of the image represented by the camera.

2. The system of claim 1 wherein the event comprises generation of pulsed x-ray energy.

3. The system of claim 1 further comprising a mirror presented at an angle to direct the light from the scintillator to the lens group.

4. The system of claim 3 wherein the mirror is a pellicle mirror.

5. The system of claim 1 wherein the lens group comprises:
a first lens group configured to receive the light from the pellicle, the first lens group comprising one or more lenses;
a first doublet lens configured to receive the light from the first lens group;
a second doublet lens configured to receive the light from the first lens group, such that the first doublet lens can move relative to the second doublet lens.

6. The system of claim 1, further comprising a shutter configured to selectively establish an aperture to electively pass and block light traveling from the lens group to the camera.

7. The system of claim 6 wherein the shutter comprises a mechanical shutter.

8. The system of claim 6 wherein the scintillator comprises a LYSO scintillator.

9. The system of claim 6 wherein the scintillator comprises a Csl(Tl) scintillator.

10. The system of claim 1 wherein the lens group is telecentric.

11. A method for recording an event comprising:
receiving energy from an event at a scintillator, the scintillator converting the energy to a light at a visible wavelength;
receiving the light along a light path axis at a first lens group, the first lens group comprising one or more fixed lenses;
presenting the light from the first lens group to first zoom lens element and a second zoom lens element, at least one of the first zoom lens element or a second zoom lens element movable in the direction of the light path axis;
receiving the light at an image plane of a camera, the light forming an image on the image plane such that the movement of the first zoom lens element or a second zoom lens element adjusts the size of the image on the image plane; and
recording the image with a camera.

12. The method of claim 11 wherein the energy comprises x-ray energy and the scintillator converts the x-ray energy to a visible wavelength.

13. The method of claim 11 wherein at least one of the first zoom lens element and the second zoom lens element comprise a lens element.

14. The method of claim 11 wherein the scintillator consisting of a LYSO scintillator or a Csl(Tl) scintillator.

15. The method of claim 11 further comprising reflecting the light from the scintillator into the first lens group with a pellicle mirror.

16. The method of claim 11, further comprising actuating a shutter to selectively present the light to the camera.

17. An imaging system for use with a pulsed radiographic x-ray source comprising:
a first lens group configured to receive light, the light representing x-ray energy;
a second lens group having zoom capability, the second lens group having at least one lens that is moveable in relation to one or more other lenses in the second lens group;
a shutter to selectively pass the light;
a camera configured to capture the light to form an image, the image representing x-rays from the radiographic x-ray source.

18. The imaging system of claim 17 wherein the light is generated by a scintillator which generates light in response to contact by x-ray radiation.

19. The imaging system of claim 17 wherein the camera comprises a 62 mm CCD camera and is movable along an axis of the light from the second lens group.

20. The imaging system of claim 17 wherein the first lens group and the second lens group establish a telecentric lens system.
